(19) United States
(12) Patent Application Publication
(10) Pub. No.: US 2009/0104275 A1
(43) Pub. Date: Apr. 23, 2009

(54) Nanoencapsulation of Proteins

(76) Inventor: ALEXANDER GRINBERG, SAN FRANCISCO, CA (US)

Correspondence Address: ALEXANDER GRINBERG MD, 600 15TH AVENUE, SAN FRANCISCO, CA, 94118, US

(21) Appl. No.: 11/563,012

(22) Filed: Nov. 23, 2006

(51) Int. Cl. A61K 9/14; C07K 14/62; A61K 38/28

(52) U.S. Cl. 424/497; 530/304; 514/3

(57) ABSTRACT

The protein encapsulation via entrapping protein in CaCO₃ microparticles followed by polymeric shell deposition can be used for vaccination based on protein antigen, and in particular rPA 102.

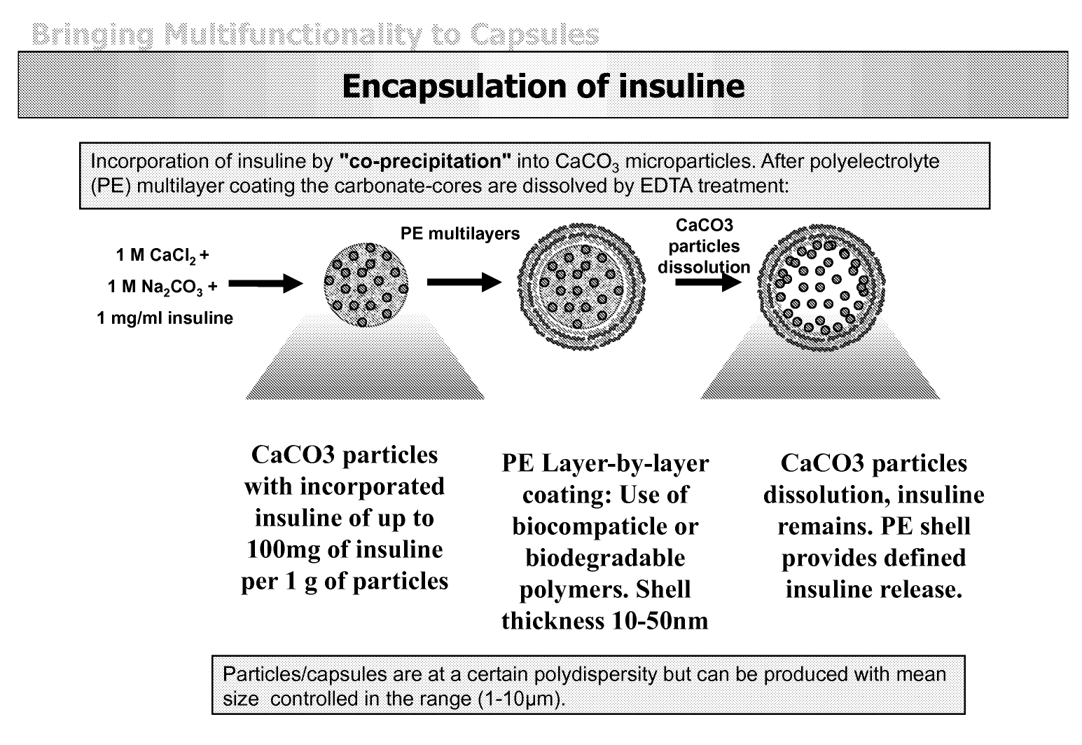

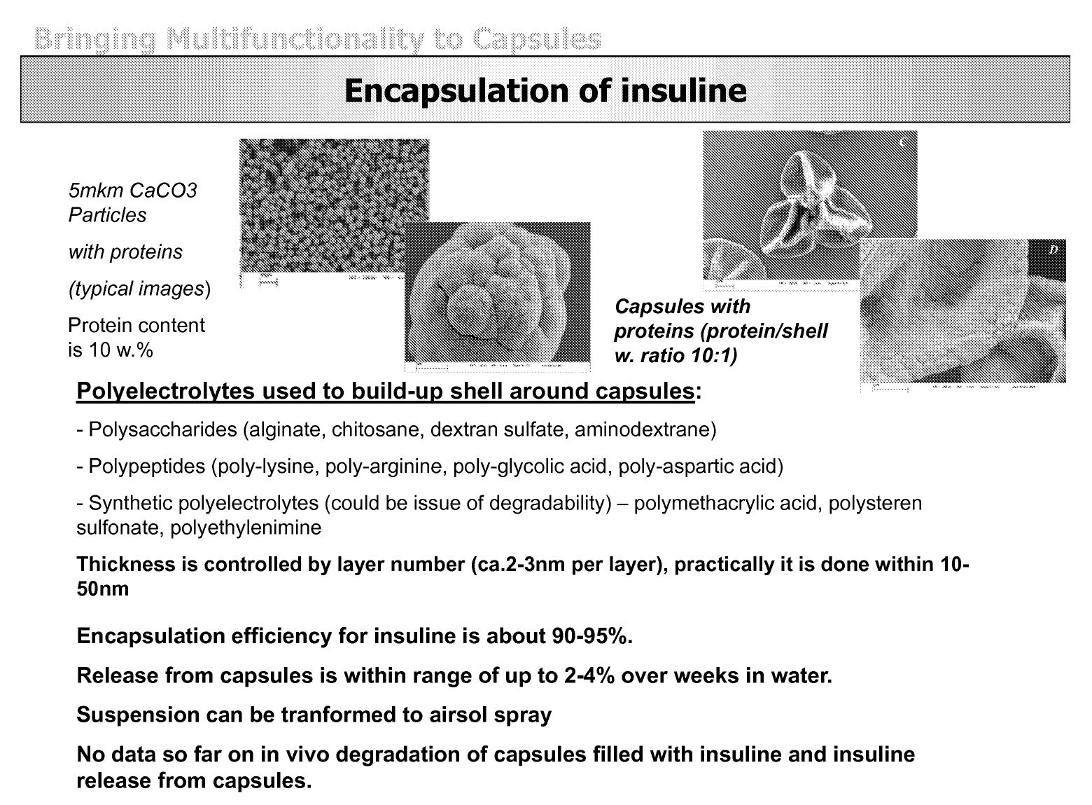

### DESCRIPTION OF TECHNOLOGY PRINCIPALS

The method of nanoencapsulation of proteins and its mixtures in polyelectrolyte microcapsules utilizes porous calcium carbonate microparticles (could be fabricated of 2-10 micron with fine size distribution) as microscopic supports for layer-by-layer (LbL) polyelectrolyte (PE) assembling via charge interaction of alternating positive and negative charged PEs. These PE multilayers (thickness, composition) determine shell of capsules and could tuned in permeability, functionality (optically and magnet addressing), stability and degradation. Range of used PEs involved synthetic and natural charged polymers (including polysaccharides and polypeptides).

Two different ways were used to prepare protein-loaded CaCO3 microparticles:
(i) physical adsorption—adsorption of proteins from the solutions onto preformed CaCO3 porous microparticles, and
(ii) co-precipitation—protein capture by CaCO₃ microparticles in the process of growth from the mixture of aqueous solutions of CaCl₂ and Na₂CO₃. amount of encapsulated materials could reach 100 μg per 1 mg of CaCO₃ and encapsulation efficiency close to 100%.

The procedure of nanoencapsulation is very mild and involved no chemical treatment, but only physical capturing. CaCO₃ particles could be dissolved at very mild condition leaving protein inside capsules. No change of protein conformation or lost of activity.

The advantage of the suggested approach is the possibility to control easily the concentration of protein inside the microcapsules and to tune release (action) time of vaccine.

Cost of technology is rather low and includes mainly costs of degradable polymers and actually compounds to be encapsulated and involved man-power. Easily done in lab scale up-to volume in liters, but could be scaled-up to larger amount.

1. Incorporation of insuline by co-precipitation into CaCO3 microparticles by mixing insulin, NaCO₃ and CaCl₂. The formed particles of CaCO₃ contain insulin in amount up to 20 w. %

2. Particles size of formed CaCO₃ particles with insulin can be controlled by stirring speed, shape of vessel and/or volume added while mixing insulin, NaCO₃ and CaCl₂. Size of the particles can be varied in range of 0.5-10 microns.

3. 1. and 2. can be done in combination of insulin and other additives co-precipitating into CaCO₃ particles.

4. Polymer shells with defined properties such as thickness, compatibility, degradation and other tailored functionality—such as magnetic or fluorescent activation—can be assembled over these CaCO₃ particles with insulin by means of layer-by-layer assembly of polyelectrolytes, interfacial adsorption, interfacial complexation, surface induced polymer synthesis, or a combined approach the where layer-by-layer method is combined with others.

5. Extraction of CaCO₃ via Ca-chelating agents or lowing pH leads to the formation of purely polymeric capsules containing insulin encapsulated in defined amount. Thus, w. % of insulin could be enriched up to 90%

6. Polymer capsules made as described in claims 4 and 5 may contain more components than just insulin in the same capsule.

7. After dissolving CaCO3 particles with Ca-chelating agents, polymeric capsules with retained insulin remain.

8. The polymer shell controlling insulin release can be engineered in a way that allows portion-like release of insulin so that different sorts of capsules release insulin at different times.

9. CaCO3 templating capsules filled with insulin or other proteins can be induced via spraying/inhalation to patient.
